(19) United States
(12) Patent Application Publication
(10) Pub. No.: US 2012/0071965 A1
(43) Pub. Date: Mar. 22, 2012

(54) IMPLANTABLE GRAFT CONNECTOR

(76) Inventors: Michael A. Longo, Glenmoore, PA (US); Michael Paris, Lansdale, PA (US); Adam Dakin, Blue Bell, PA (US)

(21) Appl. No.: 13/236,273

(22) Filed: Sep. 19, 2011

Related U.S. Application Data
(60) Provisional application No. 61/383,922, filed on Sep. 17, 2010.

(51) Int. Cl. A61F 2/82

(52) U.S. Cl. 623/1.35

(57) ABSTRACT

A connector for fluidically connecting a graft to a patient's natural vessel to enable fluid to flow through the graft into the vessel. The connector comprises a main conduit having opposing ends each configured to be implanted in the vessel; and a branch conduit having a first end integral with the main conduit and a second end connectable to the graft, wherein the branch conduit extends at angle from the main conduit at a point between the opposing ends of the main conduit.

### CROSS-REFERENCE TO RELATED APPLICATIONS

This application is a non-provisional of U.S. Provisional Application No. 61/383,922, entitled “Implantable Graft Connector,” filed on Sep. 17, 2010. The entire disclosure and contents of the above application are hereby incorporated by reference.

### BACKGROUND

1. Field of the Invention

The present invention relates generally to implantable grafts, and more particularly, to an implantable graft connector.

2. Related Art

The mammalian body has numerous body channels or vessels that have a lumen through which fluid is carried. In certain circumstances, it is necessary or desirable to connect a graft to such body vessels so as to facilitate the flow of fluid from the graft into the body vessel. Grafts that may be connected to a patient's vessel are either a biological graft or a synthetic graft. Biological grafts are often classified as either an autograft or an allograft. An autograft is a graft that is taken from another site within the patient, while an allograft is a graft take from another patient. In contrast, synthetic grafts are manufactured from a material such as dacron or polytetrafluroethylene (PTFE).

An anastomosis is commonly performed to connect a body vessel to a graft. To do so, according to one conventional approach, a surgeon delicately sews the body vessel to the graft. This procedure requires the surgeon to take care not to suture too tightly so as to tear the delicate tissue, nor to suture too loosely so as to permit leakage of fluid from the anastomosis. In addition to creating a surgical field in which it is difficult to see, leakage of fluid from the anastomosis can cause serious acute or chronic complications, which may be fatal. In addition to the inherent inconsistencies in suture tightness, incision length, placement of the suture, stitch size, and reproducibility, suturing an anastomosis can be very time consuming This difficulty is compounded by the relatively small dimensions of the vessels involved or the diseased state of the vessel.

The patency of an anastomosis contributes to a successful procedure, both by acute and long-term evaluation. Patency may be compromised due to technical, biomechanical or pathophysiological causes. Among the technical and biomechanical causes for compromised patency are poorly functioning anastomoses due to, for example, poor technique, trauma, thrombosis, intimal hyperplasia or adverse biological responses to the anastomosis. Improperly anastomosed vessels may lead to leakage, create thrombus and/or lead to further stenosis at the communication site, possibly requiring re-operation or further intervention. As such, forming an anastomosis is a critical procedure in bypass or arteriovenous (AV) fistula surgery, requiring precision and accuracy on the part of the surgeon.

### SUMMARY

In one aspect of the present invention, a connector for fluidically connecting a graft to a patient's natural vessel to enable fluid to flow through the graft into the vessel is provided. The connector comprises a main conduit having opposing ends each configured to be implanted in the vessel; and a branch conduit having a first end integral with the main conduit and a second end connectable to the graft, wherein the branch conduit extends at an angle from the main conduit at a point between the opposing ends of the main conduit.

In another aspect of the present invention, a kit for connecting a graft to a patient's vessel is provided. The kit comprises a main conduit implantable in the vessel and having opposing ends each configured to be secured to the vessel; a branch conduit having a first end attached to the main conduit and a second end connectable to the graft, wherein the branch conduit extends at an angle from the main conduit at a point between the opposing ends of the main conduit; and one or more attachment devices configured to secure the ends of the main conduit to the vessel.

### BRIEF DESCRIPTION OF THE DRAWINGS

Embodiments of the present invention are described below with reference to the attached drawings, in which:

FIG. 1 is a side view of an implantable graft connector, in accordance with embodiments of the present invention;

FIG. 2 is a side view of another implantable graft connector, in accordance with embodiments of the present invention;

FIG. 3 is a side view of a still other implantable graft connector, in accordance with embodiments of the present invention;

FIG. 4 is a cross-sectional view of an implantable graft connector, in accordance with embodiments of the present invention; and

FIG. 5 is a perspective view of the graft end of an implantable graft connector shown adjacent the connector end of a vascular graft, in accordance with embodiments of the present invention.

### DETAILED DESCRIPTION

Aspects of the present invention are generally directed to an graft connector configured to fluidically connect a graft, such as a vascular graft, to a patient's natural vessel so as to enable the flow of fluid from the graft into the patient's vessel. The graft connector comprises a main conduit having opposing ends each configured to be implanted in the vessel. The graft connector also comprises a branch conduit having a first end that is integral with the main conduit at point between the opposing ends of the main conduit. The branch conduit extends from the main conduit at an angle, and the second end of the branch conduit is connectable to the graft. More particularly, fluid flows through the main conduit in one direction, while fluid flows from the branch conduit into the main conduit. The junction of the two fluid flows, and hence the connection of the main and branch conduits, is at an acute angle.

As would be appreciated, embodiments of the present invention may be used to connect a variety of grafts to many different vessels. For ease of description, embodiments of the present invention will be described with reference to the connection of a vascular graft to a patient's blood vessel.

As noted, the graft connector in accordance with embodiments of the present invention provides a pathway for the flow of blood from the vascular graft into the patient's blood vessel. In certain embodiments, the connector is designed to decrease the velocity and turbulent flow of blood as it enters the vessel, thereby reducing the potential for damage, such as intimal hyperplasia, to the vessel. More specifically, as high velocity blood flow exits the graft and enters the vessel, the blood flow is absorbed by the main conduit, rather than the back wall of the native vessel. By directing this high velocity flow into the main conduit, thickening of the tunica intima of the blood vessel (intimal hyperplasia) is substantially reduced.

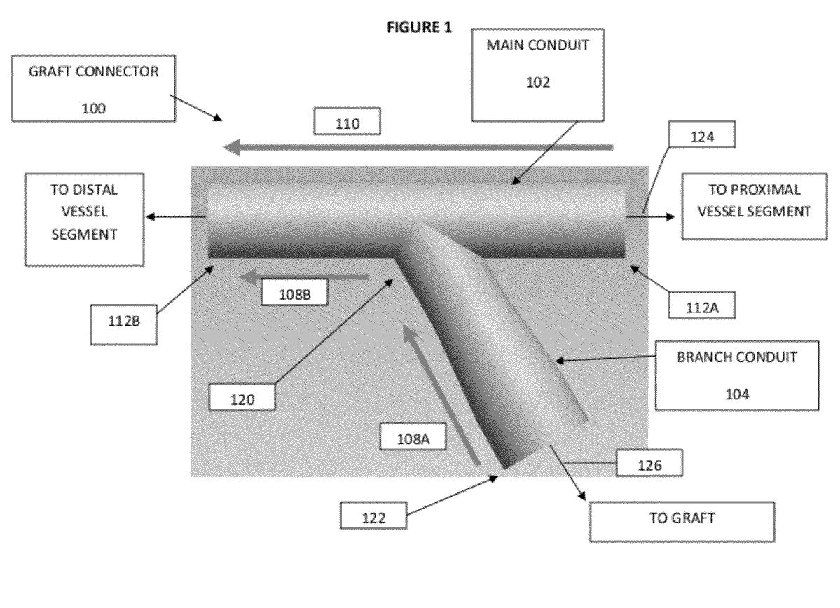

FIG. 1 is a side view of an implantable graft connector 100 in accordance with embodiments of the present invention. As shown, graft connector 100 has a main conduit 102, and a branch conduit 104. Main conduit 102 comprises a generally tubular structure having opposing ends, and having a lumen extending therethrough. That is, main conduit 102 has an outer surface that forms a closed curve, and has a cross-sectional shape that is substantially circular to oval. In the embodiment of FIG. 1, the cross-sectional shape of main conduit 102 substantially matches the cross-sectional shape of the patient's vessel in which the conduit is implanted. For ease of illustration, the patient's vessel is not shown in FIG. 1.

When main conduit 102 is positioned in the vessel, the conduit may be secured to the vessel using attachment devices. Exemplary attachment devices are described in further detail below.

As noted, graft connector 100 further comprises branch conduit 104 extending at an angle from main conduit 102. Similar to main conduit 102, branch conduit 104 comprises a generally tubular structure having a lumen therein. The outer surface of branch conduit 104 also forms a closed curve, and that has a cross-sectional shape that is substantially circular to oval. Additionally, the proximal region 120 of branch conduit 104 is integral with main conduit 102 so that the junction between the conduits does not allow the ingress or egress of fluids.

As shown in FIG. 1, branch conduit 104 has a distal region 122 configured to be attached to a graft (not shown). In certain embodiments, distal region 122 has a cross-sectional shape that substantially matches the cross-sectional shape of the graft.

In the embodiments of FIG. 1, the cross-sectional shape of proximal region 120 (i.e. the region most proximate to main conduit 102) is smaller than the cross-sectional shape of distal region 122. In a specific embodiment of FIG. 1, branch conduit 104 is tapered towards main conduit 102 so that the cross-section of the conduit continually decreases from a first shape at distal region 122 to a second shape at 120. In another embodiment, the change in the cross-sectional shape comprises one or more step changes.

As previously noted, main conduit 102 is implanted in a patient's vessel. It would be appreciated that in certain embodiments main conduit 102 may be implanted by creating an end-to-end anastomosis of the vessel, or by making a arteriotomy or venotomy. In embodiments in which an end-to-end anastomosis is created, the patient's vessel would be severed to form two separate segments. Distal end 112B of main conduit 102 is implanted in the distal segment of the patient's vessel, while proximal end 112A of the main conduit is implanted in the proximal vessel segment. As described further below, proximal and distal ends 112 may each be secured to the respective proximal and distal vessel segments. In embodiments using a arteriotomy or venotomy, proximal and distal ends 112 are sequentially inserted into an incision and a substantial portion of main conduit 102 is positioned in the vessel. Similar to the end-to-end anastomosis embodiments, proximal and distal ends 112 may be secured to the respective proximal and distal vessel segments. Regardless of whether only the ends of main conduit 102, or a substantial portion of the main conduit is positioned in the vessel, the main conduit is referred to as herein being implanted or positioned in the vessel.

When main conduit 102 is positioned in the patient's vessel, and when the graft is attached to distal end 122, blood may flow from the graft through graft connector 100 into the patient's vessel. The flow of blood between the graft and the vessel via connector 100 is illustrated by arrows 108. Because, as noted above, main conduit 102 has a tubular structure, the force of blood flowing into the main conduit is absorbed by the conduit and does not damage the patient's vessel. Additionally, in certain circumstances, blood may still flow from the proximal vessel segment through conduit 102 into the distal vessel segment. This flow of blood is illustrated by arrow 110.

As shown in FIG. 1, branch conduit 104 extends at an angle from a point between the opposing ends of main conduit 102. That is, central axis 126 of branch conduit 104 is offset from central axis 124 of main conduit 102 by an angle. More particularly, fluid flow 110 through main conduit 102 is in one direction, while fluid flow 108 flows from branch conduit 104 into the main conduit. The junction of the two fluid flows, and hence the connection of the main an branch conduits 102, 104, is at an acute angle.

Although the selected angle may vary, in certain circumstances, the angle and/or the size and cross-sectional shapes of conduits 102, 104, may be selected to one or more to reduce the force applied to main conduit 102 by the blood flowing from branch conduit 104, reduce the velocity of the blood flow and/or decrease the turbulence of the blood flow.

As would be appreciated, graft connector 100 may be formed from a number of different materials. In certain embodiments, graft connector 100 is formed from a non-thrombogenic, biostable polymer.

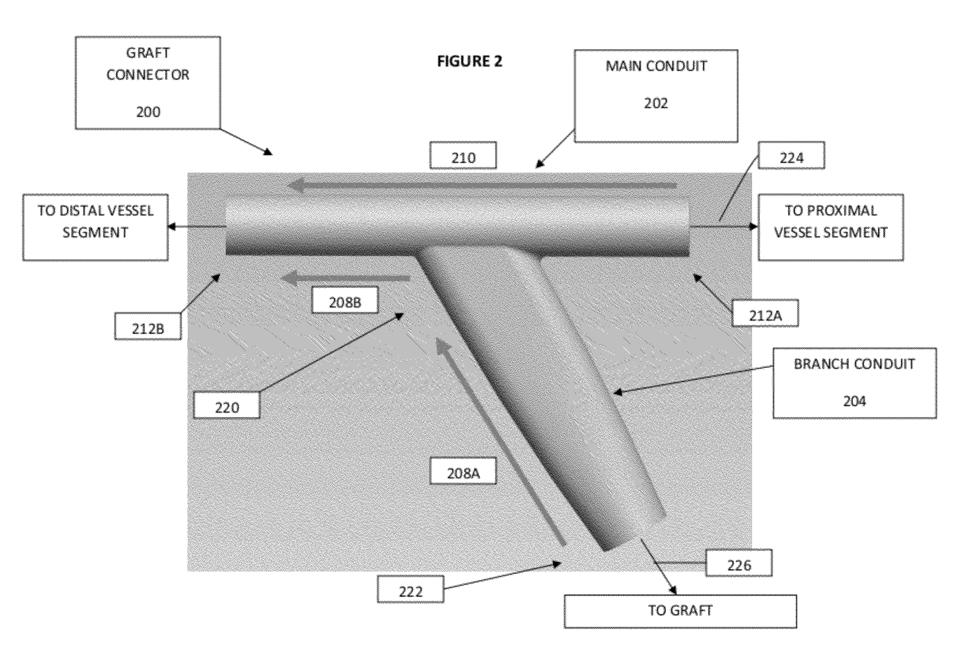

FIG. 2 is a side view of another implantable graft connector 200, in accordance with embodiments of the present invention. For ease of description, features of graft connector 200 that are similar to features of graft connector 100 are shown using like numbers. Accordingly, certain elements previously described above with reference to FIG. 1 will not be described in detail with reference to FIG. 2.

As shown, graft connector 200 comprises a main conduit 202, and a branch conduit 204. Similar to the embodiments of FIG. 1, main conduit 202 opposing ends, and has a cross-sectional shape that substantially matches the cross-sectional shape of the vessel. For ease of illustration, the patient's vessel, in which main conduit 202 is implantable, is not shown in FIG. 2.

As noted, graft connector 200 further comprises branch conduit 204 extending from main conduit 202. Similar to the embodiments of FIG. 1, branch conduit 204 has a proximal region 220 integrated with main conduit 202 so that the junction between the conduits prevents the ingress or egress of fluids. Furthermore, distal region 222 of branch conduit 204 is configured to be attached to a graft (not shown). In certain embodiments, distal region 222 has a cross-sectional shape that substantially matches the cross-sectional shape of the graft.

As shown in FIG. 2, the cross-sectional shape of proximal region 220 is larger than the cross-sectional shape of distal region 222. In a specific embodiment of FIG. 2, branch conduit 204 is tapered away from main conduit 202 so that the cross-sectional shape of the conduit continually decreases from a first cross-sectional shape at proximal region 220 to a second cross-sectional shape at distal region 222. In another embodiment, the change in the cross-sectional shape comprises one or more step changes.

Similar to the previously described embodiments, when main conduit 202 is positioned in the patient's vessel, and when the graft is attached to distal end 222, blood may flow from the graft through graft connector 200 into the patient's vessel, as illustrated by arrows 208. Because, as noted above, main conduit 202 is a tubular structure, the force of blood flowing into the main conduit is absorbed by the conduit and does not damage the patient's vessel. In certain circumstances, blood may still flow, as illustrated by arrow 210, from the proximal vessel segment through conduit 202 into the distal vessel segment.

Additionally, branch conduit 204 extends from main conduit 202 at an angle. That is, central axis 226 of branch conduit 204 is offset from central axis 224 of main conduit 202 by an angle. As previously noted, the angle may vary and, in certain circumstances, the angle and/or the size and cross-sectional shapes of conduits 202, 204, may be selected to one or more of reduce the force applied to main conduit 202 by the blood flowing from branch conduit 204, reduce the velocity of the blood flow and/or decrease the turbulence of the blood flow.

FIGS. 1 and 2 illustrate embodiments in which the cross-sectional shape of branch conduits 104, 204 changes along the length thereof. It would be appreciated that the change in shape is merely illustrative and embodiments in which the cross-sectional shape of branch conduits 104, 204 remain substantially consistent are within the scope of the present invention.

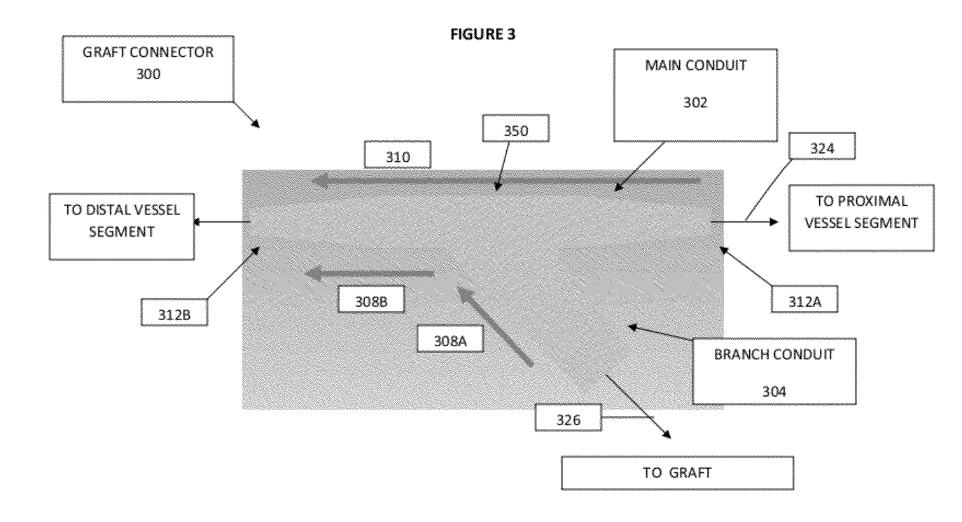

FIG. 3 is a side view of another implantable graft connector 300, in accordance with embodiments of the present invention. For ease of description, features of graft connector 300 that are similar to features of graft connector 100 are shown using like numbers. Accordingly, certain elements previously described above with reference to FIG. 1 will not be described in detail with reference to FIG. 3.

As shown, graft connector 300 comprises a main conduit 302, and a branch conduit 304. For ease of illustration, the patient's vessel, in which main conduit 302 is implantable, is not shown in FIG. 2.

As show in FIG. 3, main conduit 302 has proximal and distal regions 312, and a central region 350. In these specific embodiments, the cross-sectional shape of proximal and distal regions 312 is smaller than the cross-sectional shape of central region 350. In a specific embodiment of FIG. 3, regions 312 of main conduit 302 are each tapered away from central region 350 so that the cross-section of the conduit continually decreases from a first shape at the central region to second, smaller shapes and the ends of the conduit. In another embodiment, the change in the cross-sectional shape comprises one or more step changes.

As noted elsewhere in connection with other embodiments, graft connector 300 further comprises branch conduit 304 extending from, and integrated with, main conduit 302. Similar to the embodiments of FIG. 1, branch conduit 304 comprises a generally tubular structure having opposing ends. Branch conduit 304 has a cross-sectional shape substantially matching the cross-sectional shape of the graft that is to be attached thereto.

When main conduit 302 is positioned in the patient's vessel, and when the graft is attached to branch conduit 304, blood may flow from the graft through graft connector 300 into the patient's vessel, as illustrated by arrows 308. Because, as noted above, main conduit 302 is a tubular structure that receives the fluid flow from the graft, the force of blood flowing into the main conduit is absorbed by the conduit and does not damage the patient's vessel. In certain circumstances, blood may still flow, as illustrated by arrow 310, from the proximal vessel segment through conduit 302 into the distal vessel segment.

Additionally, branch conduit 304 extends from main conduit 302 at an angle. That is, central axis 326 of branch conduit 304 is offset from central axis 324 of main conduit 302 by an angle. As previously noted the angle may vary and, in certain circumstances, the angle and/or the size and cross-sectional shapes of conduits 302, 304, may be selected to one or more of reduce the force applied to main conduit 302 by the blood flowing from branch conduit 304, reduce the velocity of the blood flow and/or decrease the turbulence of the blood flow.

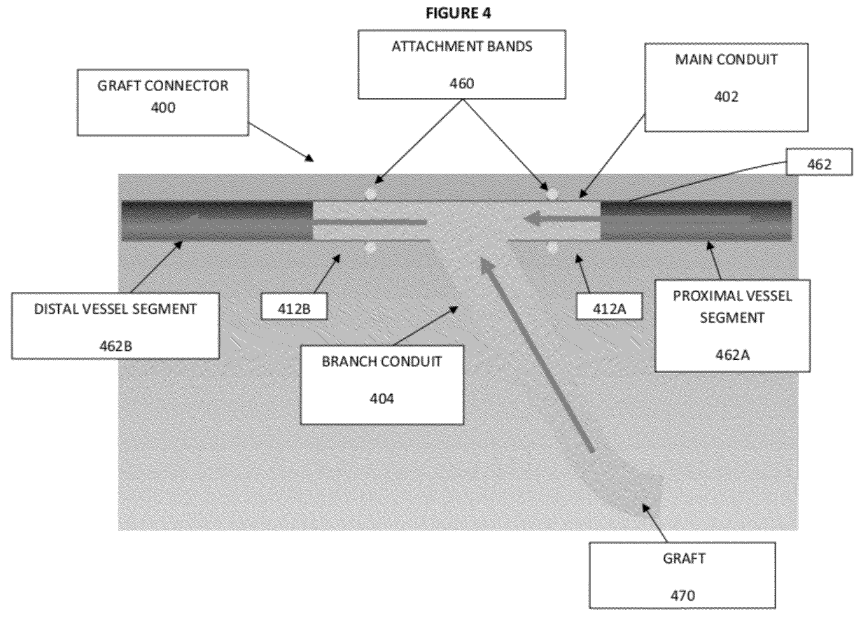

FIG. 4 is a cross-sectional view of an implantable graft connector 400, in accordance with embodiments of the present invention. For ease of description, features of graft connector 400 that are similar to features of graft connector 100 are shown using like numbers. Accordingly, certain elements previously described above with reference to FIG. 1 will not be described in detail with reference to FIG. 4.

As shown, graft connector 400 comprises a main conduit 402, and a branch conduit 404. Similar to the embodiments of FIGS. 1 through 3, main conduit 402 and branch conduit 404 are each generally tubular structures having opposing ends. In the embodiments of FIG. 4, main conduit 402 is implanted in a vessel 462, and has a cross-sectional shape that substantially matches the cross-sectional shape of vessel 462.

As shown in FIG. 4, branch conduit 404 extends from body vessel 402 at an angle, and is integrated with the main conduit. In these illustrative embodiments, branch conduit 404 is attached to graft 470 and has a cross-sectional shape that is substantially the same as the cross-sectional shape of the graft. In certain embodiments, branch conduit 404 is pre-attached to graft 470 to create a seamless transition.

As show in FIG. 4, main conduit 402 has proximal and distal regions 412 implanted in proximal and distal segments, respectively, of vessel 462. In these specific embodiments, regions 412 are each secured to vessel segments 462 by an attachment device. In the embodiments of the FIG. 4, the attachment device comprises attachment bands 460 that extend about the circumference of vessel 462 to retain the vessel wall in contact with the outer surface of main conduit 402. In the embodiments of FIG. 4, attachment bands 460 comprise a shape memory material, such as nitinol elastic bands or elastic o-rings.

FIG. 4 illustrates specific embodiments in which attachment bands 460 are used to secure vessel 462 to main conduit 402. However, it would be appreciated that other attachment devices may be used instead of, or in addition to, bands 460. For example, in certain embodiments other types of clips or sutures may be used to secure conduit 402 to vessel 462.

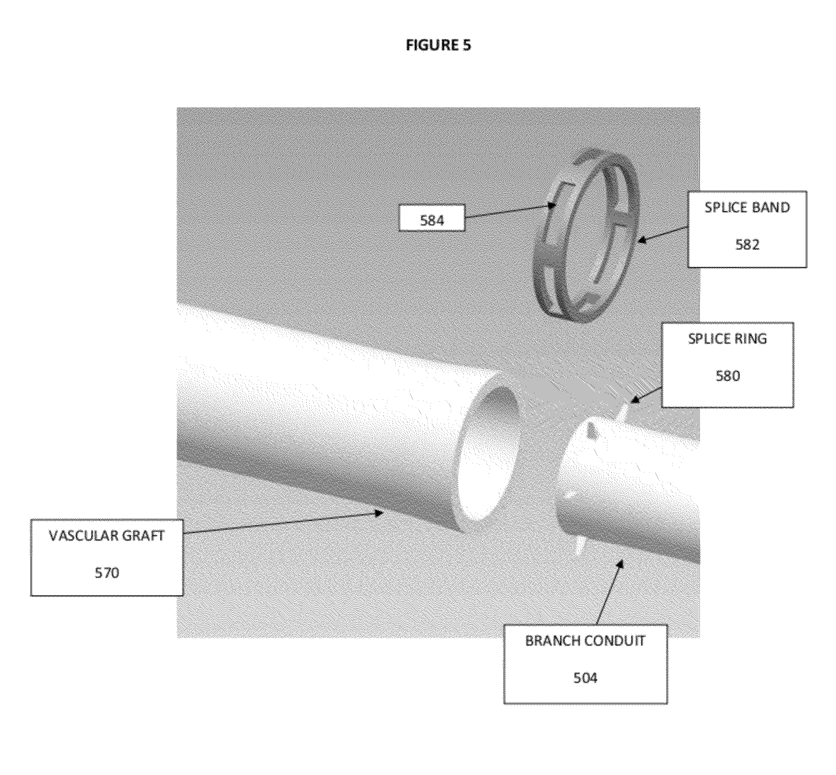

FIG. 5 is a perspective view of the distal or graft attachment end of a branch conduit 504 of a graft connector 500 (not shown). Graft connector 504 is shown adjacent the connector end of a vascular graft 570.

As previously noted, in certain embodiments a graft may be pre-attached to a branch conduit. However, in certain embodiments, it is desirable to attach the graft to the branch conduit during a surgical procedure. FIG. 5 illustrates one such system for attaching vascular graft 570 to branch conduit 504 prior to or during surgery. In these embodiments, branch conduit 504 comprises a splice ring 580 having a series of radial extension members in the form of spikes extending from the outer surface of branch conduit 504. In operation, splice band 582 is positioned around graft 570. Branch conduit 504 is inserted into graft 570 such that the spikes penetrate the wall of the graft and extend through the openings 584 in splice band 582. Splice band 582 and splice ring 580 create a robust joint between graft 570 and branch conduit 504.

All documents, patents, journal articles and other materials cited in the present application are hereby incorporated by reference.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart there from.

1. A connector for fluidically connecting a graft to a patient's natural vessel to enable fluid to flow through the graft into the vessel comprising:
a main conduit having opposing ends each configured to be implanted in the vessel; and
a branch conduit having a first end integral with the main conduit and a second end connectable to the graft, wherein the branch conduit extends at angle from the main conduit at a point between the opposing ends of the main conduit.

2. The graft connector of claim 1, wherein the branch conduit has a proximal region and a distal region, and wherein the cross-sectional shape of the proximal region is smaller than the cross-sectional shape of the distal region.

3. The graft connector of claim 2, wherein the branch conduit is tapered from the distal region to the proximal region.

4. The graft connector of claim 1, wherein the branch conduit has a proximal region and a distal region, and wherein the cross-sectional shape of the proximal region is larger than the cross-sectional shape of the distal region.

5. The graft connector of claim 4, wherein the branch conduit is tapered from the proximal region to the distal region.

6. The graft connector of claim 1, wherein the branch conduit is configured to be attached to a graft, and wherein the branch conduit has a cross-sectional shape that substantially matches the cross-sectional shape of the graft.

7. The graft connector of claim 1, wherein the main conduit has a proximal region, central region and a distal region, and wherein the cross-sectional shape of the central region is larger than the cross-sectional shapes of the proximal and distal regions.

8. The graft connector of claim 7, wherein the main conduit is tapered from the central region to the ends of the proximal and distal regions.

9. A kit for connecting a graft to a patient's vessel, comprising:
a main conduit implantable in the vessel and having opposing ends each configured to be secured to the vessel;
a branch conduit having a first end attached to the main conduit and a second end connectable to the graft, wherein the branch conduit extends at angle from the main conduit at a point between the opposing ends of the main conduit; and
one or more attachment devices configured to secure the ends of the main conduit to the vessel.

10. The kit claim 9, wherein the one or more attachment devices to secure the ends of the main conduit comprise:
shape memory attachment bands.

11. The kit of claim 10, wherein the attachment bands comprises at least one of a nitinol elastic band and a rubber o-ring.

12. The kit of claim 9, wherein the branch conduit has a proximal region and a distal region, and wherein the cross-sectional shape of the proximal region is smaller than the cross-sectional shape of the distal region.

13. The graft connector of claim 12, wherein the branch conduit is tapered from the distal region to the proximal region.

14. The graft connector of claim 9, wherein the branch conduit has a proximal region and a distal region, and wherein the cross-sectional shape of the proximal region is larger than the cross-sectional shape of the distal region.

15. The graft connector of claim 14, wherein the branch conduit is tapered from the proximal region to the distal region.

16. The graft connector of claim 9, wherein the branch conduit is configured to be attached to a graft, and wherein the branch conduit has a cross-sectional shape that substantially matches the cross-sectional shape of the graft.

17. The graft connector of claim 9, wherein the main conduit has a proximal region, central region and a distal region, and wherein the cross-sectional shape of the central region is larger than the cross-sectional shapes of the proximal and distal regions.

18. The graft connector of claim 7, wherein the main conduit is tapered from the central region to the ends of the proximal and distal regions.
